(19) United States
(12) Patent Application Publication
(10) Pub. No.: US 2020/0330703 A1
(43) Pub. Date: Oct. 22, 2020

(54) SYSTEM FOR MAINTAINING TEMPERATURE OF LIQUID MEDICINE

(71) Applicants: Matthew Carl Mosher, Hugo, CO (US); Rosemary Kinney Mosher, Hugo, CO (US)

(72) Inventors: Matthew Carl Mosher, Hugo, CO (US); Rosemary Kinney Mosher, Hugo, CO (US)

(21) Appl. No.: 16/839,126

(22) Filed: Apr. 3, 2020

Related U.S. Application Data
(60) Provisional application No. 62/836,800, filed on Apr. 22, 2019.

(51) Int. Cl. A61M 5/44; A61B 17/20; A61M 5/315; A61M 5/31

(52) U.S. Cl. CPC A61M 5/44; A61B 17/205; A61M 5/31511; A61M 5/3129; A61M 2205/3633; A61M 2202/30; A61M 2250/00; A61M 2205/3327; A61M 2205/3368

(57) ABSTRACT

A medicine disbursement system for maintaining a desired temperature of a liquid medication for an extended period of time before administration. The system includes a disburser having a plunger assembly surrounded by an insulation layer. An insulated medicine container is also operably used in cooperation with the disburser. A selective end to end arrangement between the disburser and the container provides a closed and insulated storage arrangement for both the medicine in the disburser and the medicine in the container. The operative connection of the dispenser and the container permits the medicine distribution system to be conveniently located for usage.

### CROSS REFERENCE TO RELATED APPLICATION

This application claims the benefit of co-pending U.S. Provisional Patent application Ser. No. 62/836,800 filed Apr. 22, 2019.

### TECHNICAL FIELD

The present disclosure is directed to a medicine disbursement system. More particularly, the present disclosure is directed to a system and method for maintaining a temperature of a liquid medication, such as a vaccine, for an extended period of time before administration.

### BACKGROUND

Many medications are temperature-sensitive, and exposure of these medications to extreme temperatures can damage or destroy the effectiveness of these mediations. Medications, such as vaccines, cannot be frozen, and also cannot have a temperature above a certain point for more than a particular length of time. Certain medications need to be administered in a location which has limited or no access to electricity. Such areas may include, for example, ranches, remote locations, or other areas where temperature extremes may occur.

While the art includes examples of different liquid medicine delivery apparatus, there is a need for a system and method for maintaining a desired temperature of a liquid medication for an extended period of time before injection. Further, a need exists for the system and method to be usable in remote locations such as, for example, a cattle ranch.

### SUMMARY

The present invention is directed towards a medicine disbursement system for maintaining a desired temperature of a liquid medication for an extended period of time before administration,

The preferred embodiment of the medicine disbursement system includes a medicine disburser which has a hand activator and a plunger assembly. The medicine disburser includes a double walled outer exterior with the two walls being separated from each other to form an insulation layer. The insulation layer that exists between the two walls of the medicine disburser is preferably formed as a sealed vacuum insulation layer or space.

The medicine disburser also includes a needle that is operably connected to the plunger assembly. The plunger assembly is surrounded by the insulation layer, and the plunger assembly forces medicine through the needle for delivery into a desired target. The plunger assembly and insulation layer provide a closed insulation compartment and path for maintaining the temperature of the liquid medicine for an extended period of time before delivery of the medication through the needle. The medicine disburser further includes a disbursement tracker for monitoring the amount of medicine being dispensed and the temperature of the disbursed medicine.

The medicine distribution system of the present invention includes an insulated medicine container which may be used independently or in cooperation with the medicine disperser. The medicine container has a double walled exterior with the two walls being separated from each other to form an insulation layer. The insulation layer is preferably formed as a sealed vacuum insulation layer or space.

Prior to use, the medicine disburser may be operatively connected end to end relative to the medicine container by inserting the needle of the disburser through an end on the medicine container. The end to end arrangement between the disburser and container provides a closed and insulated storage arrangement for both the medicine in the dispenser as well as the medicine in the container. Further, when inserted into the medicine container, the cleanliness of the needle is protected and the stored needle is shielded from environmental temperature extremes. Moreover, the operative connection of the dispenser to the container permits the medicine disbursement system to be conveniently located for usage.

The medicine disbursement system of the present invention is selectively securable at any desired location along a fence line. This provides the operator with the ability to conveniently position the medicine disbursement system while the operator is preparing to inject medicine into the target.

These and other features and advantages of the present invention will be better understood with reference to the following description and appended claims. The accompanying drawings, which constitute a part of the present specification, illustrate the invention and, together with the written description, serve to explain the principles of the inventive product and method.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a schematic view of an exemplary use of the medicine disbursement system of the present invention.

FIG. 2 is a perspective view of the medicine disburser of the present invention and a cutaway exploded view of the insulation layer.

FIG. 3 is a perspective view of the medicine container of the present invention with a cutaway view of the insulation layer.

FIG. 4 is a perspective view of the medicine disbursement system of the present invention.

FIG. 5 is a perspective view of the medicine disburser with a cutaway view of the plunger and sensor.

FIG. 6 is a perspective view of a releasable attaching mechanism between the medicine disburser and the medicine container.

### DETAILED DESCRIPTION

Reference will now be made in detail to the inventive product and method of the present invention. It will be apparent to one of ordinary skill in the art that various modifications and variations can be made in the present invention without departing from the scope or spirit of the invention. Thus, it is intended that the present invention covers such modifications and variations as fall within the scope of the appended claims and their equivalents.

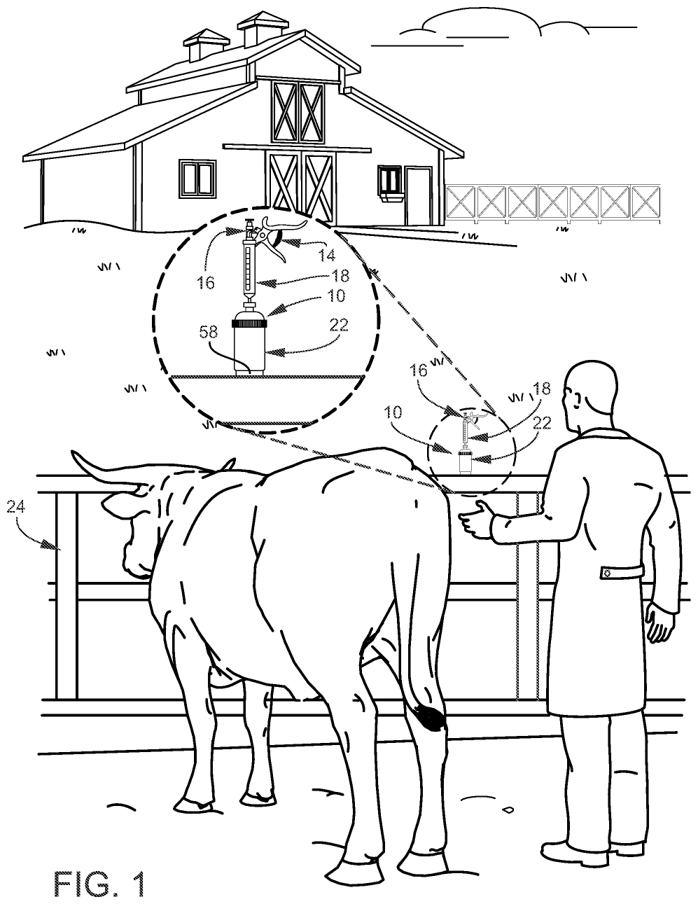

FIG. 1 is a schematic diagram, in accordance with the present invention, of an exemplary use of a medicine disbursement system 10. In the exemplary diagram, medicine disbursement system 10 is used for administering medication to livestock. System 10 may be used to administer medications in locations which have limited or no access to electricity such as, for example, ranches, remote locations, or other areas where temperature extremes may occur.

Initially, the medicine to be administered is kept at a desired temperature in a controlled environment such as, for example, a refrigeration unit. The medicine is then moved from the controlled environment to an area such as shown, for example, in FIG. 1 for administration to livestock, other animals, or people.

Medicine disbursement system 10 is a closed system for providing extended temperature control for medicine. System 10 is directed towards maintaining a desired temperature of a liquid medication for an extended period of time before administration. The closed system provides temperature insulation for the medicine, and the system is usable for environmental temperature changes in either hot or cold temperature environments.

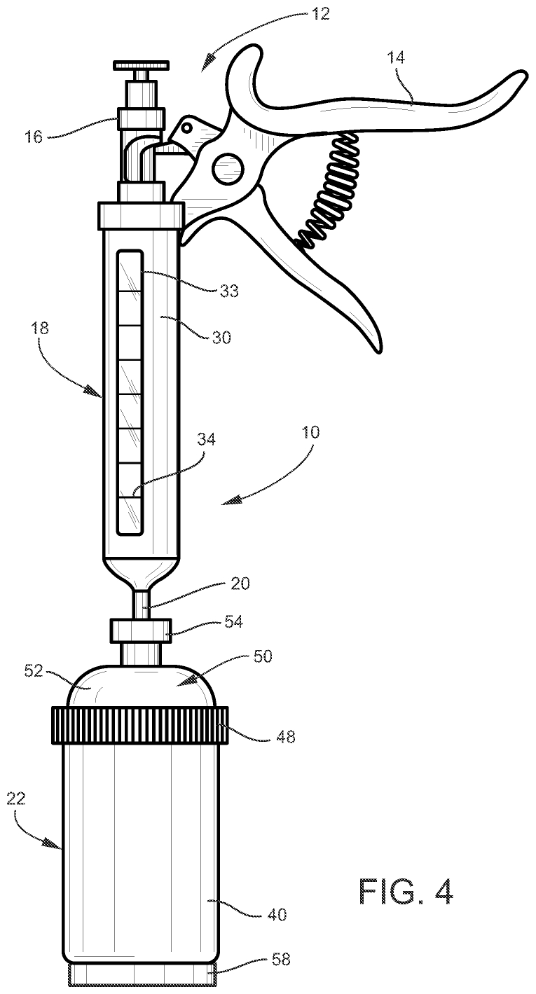

Referring to FIGS. 1-4, the medicine disbursement system 10 and its component elements are illustrated. As shown in FIGS. 1 and 4, system 10 includes a manual control assembly 12 (FIG. 4) that consists of a hand activator 14 and an adjustable plunger assembly 16. The hand activator 14 and plunger assembly 16 are operatively connected to a medicine disbursement assembly 18.

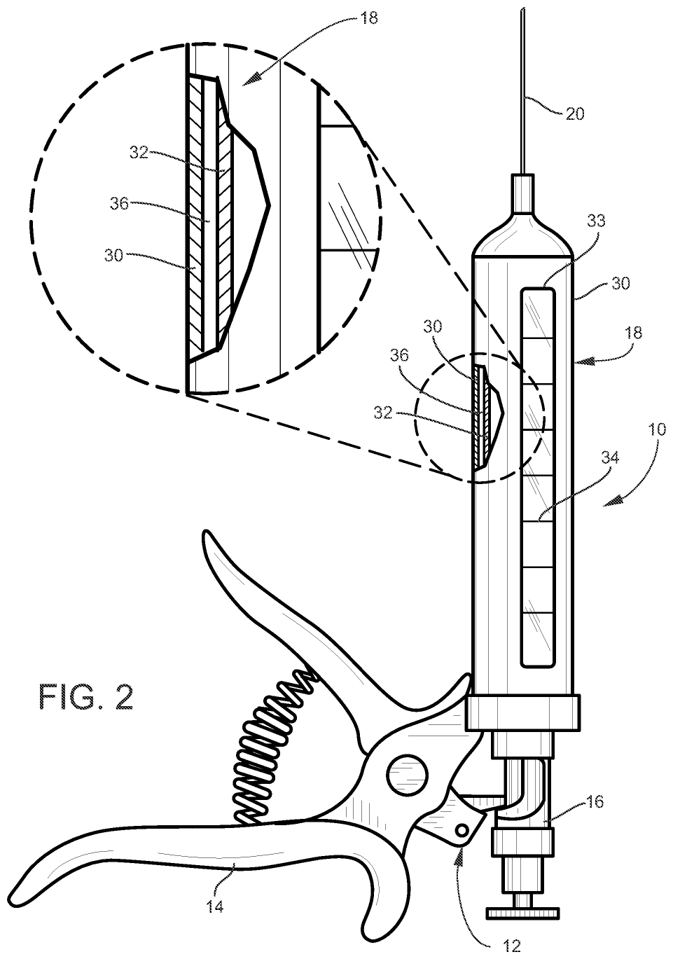

As shown in FIG. 2, medicine disbursement assembly 18 includes a needle 20 which is operably connected to plunger assembly 16. Needle 20 may be inserted into a medicine container assembly 22 as shown in FIGS. 1 and 4. The medicine assembly 18 and medicine container assembly 22 may be used independently of each other or in cooperation with each other as will be further described.

Referring again to FIG. 1, the medicine disbursement assembly 18 and medicine container 22 may be selectively connected at any desired location along a fence line 24. This allows the operator to provide temporary storage for the medicine and also provides the operator with convenient access to either the medicine disbursement assembly 18 or the medicine container 22. Thus, the ability to connect to a fence line 24 at a desired location provides the operator with the ability to conveniently position the medicine disbursement system 10 while the operator is injecting the medicine into the target.

FIG. 2 illustrates the medicine disbursement assembly 18 of the present invention which includes a double walled exterior having an outer wall 30 and an inner wall 32. Walls 30 and 32 are separated by an insulation layer or space 36. The insulation layer 36 that exists between the two walls 30 and 32 is preferably formed as a sealed vacuum insulation layer or space. Alternatively, the walls 30 and 32 may be made of known insulating materials and/or the insulation layer 36 may consist of or include a known insulation material.

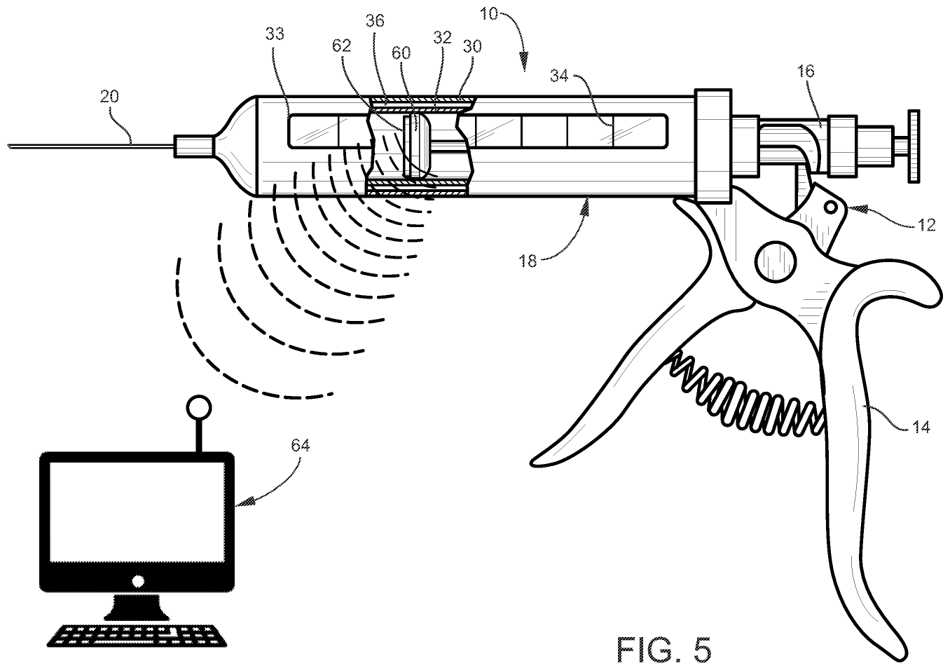

FIGS. 2 and 5 illustrate the adjustable plunger assembly 16 and double walled insulation layer 36. The plunger 60 (FIG. 5), of the plunger assembly 16, is surrounded by the insulation layer 36 during the linear movement of the plunger 60. The plunger assembly 16 forces medicine through the syringe 20 for delivery into a desired target. Plunger 60 and insulation layer 36 provide a closed insulation compartment and pathway for maintaining the temperature of the liquid medicine for an extended period of time before delivery of the medicine through the needle 20 and into the desired target.

FIGS. 2 and 4-6 illustrate the medicine disbursement assembly 18 having a vision portion 33 and equally spaced measuring indicators 34. Vision portion 33 may be made of a clear plastic to allow an operator to visually observe the movement of medicine resulting from the linear movement of the plunger 60. As is known, the manual control assembly 12, including the hand activator 14 and the plunger assembly 16, is activated by squeezing the activator 14 thereby linearly moving the plunger 60 which results in the dispensing of medicine through the needle 20. The measuring indicators 34 are spaced to indicate the amount of medicine dispersed so that the operator can monitor the amount of medicine that is given to the recipient of the medicine.

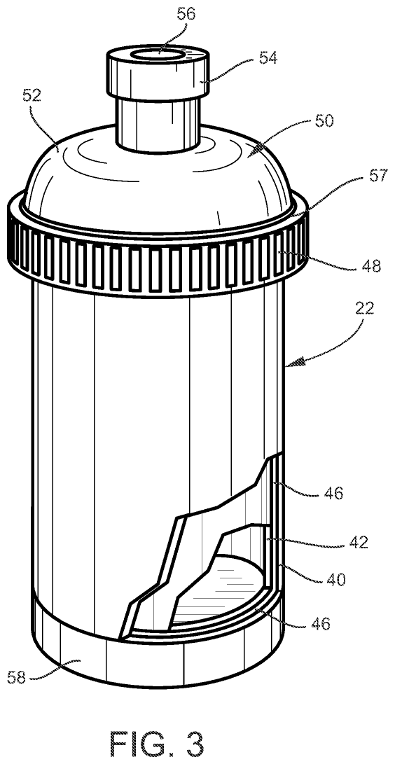

The medicine container assembly 22 is illustrated in FIGS. 3 and 4. The medicine container assembly 22 includes a double walled exterior having an outer wall 40 and an inner wall 42. Walls 40 and 42 are separated by an insulation layer or space 46. The insulation layer 46 that exists between the two walls 40 and 42 is preferably formed as a sealed vacuum insulation layer or space. Alternatively, the walls 40 and 42 may be made of known insulating materials and/or the insulation layer 46 may consist of or include a known insulation material.

One end of the medicine container 22 includes a threaded release ring 48, which releasably secures a separate cover assembly 50 to the lower body of the medicine container 22. The cover assembly 50 includes a domed base portion 52 and an end cap portion 54. The end cap portion 54 includes a dispensing and needle support area 56. A dense foam material may be used in the support area 56 for sealing the medicine container while allowing the needle 20 to be inserted into the container 22 thereby allowing medicine to be drawn through needle 20.

The preferred embodiment of the medicine container 22 is illustrated in FIG. 3. In this embodiment, the medicine is poured into the container 22 after the cover assembly 50 is removed. Another version of the medicine container 22 includes placing the medicine into a separate container that has an end similar to cover 50. In this embodiment, the existing cover 50 on container 22, as shown in FIG. 3, would be removed and the separate container of medicine would be inserted into the open body of container 22. A sealing ring 57, in cooperation with the threaded release ring 48, would provide a seal between the medicine container 22 (shown in FIG. 3) and the separate container of medicine (not shown). Both embodiments of medicine container 22 are directed towards the maintenance of a desired temperature of a liquid medication for an extended period of time before administration.

Prior to dispensing medicine, the medicine disburser 18 may be operably connected, in an end to end fashion, to the medicine container 22, by inserting the needle 20 into the end cap portion 54 of medicine container 22. Referring to FIGS. 1 and 3-4, the medicine container 22 includes an attachment device 58 for selectively securing the medicine disbursement system 10 at any desired location along a fence line. The attachment device 58 may be a magnet or any other device that is capable of releasably attaching the disbursement system 10 to a selected position along a fence line. The releasable attachment device 58 provides the operator with the ability to conveniently position the medicine disbursement system 10 while the operator is preparing to inject medicine into the target.

Referring again to FIGS. 1 and 4, the end to end arrangement between the medicine disburser 18 and the medicine container 22 provides a closed and insulated storage arrangement for both the medicine in the dispenser 18 as well as the medicine in the container 22. Further, when inserted into the medicine container 22, the cleanliness of the needle 20 is protected, and the stored needle is shielded from environmental temperature extremes. Moreover, the operative connection of the dispenser 18 to the container 22 permits the medicine disbursement system 10 to be selectively located at a desired location for usage.

Referring now to FIG. 5, a sensor 62 may be operatively connected to the plunger 60. Sensor 62 detects the temperature of the medicine and transmits the detected information to a control unit 64 for storage. The position of the plunger 60 is also detected by sensor 62, and the detected position information is transmitted to control unit 64. For position detection, the indicators 34 are made of a detectable material so that the sensor 64 is able to detect the position of the plunger 60.

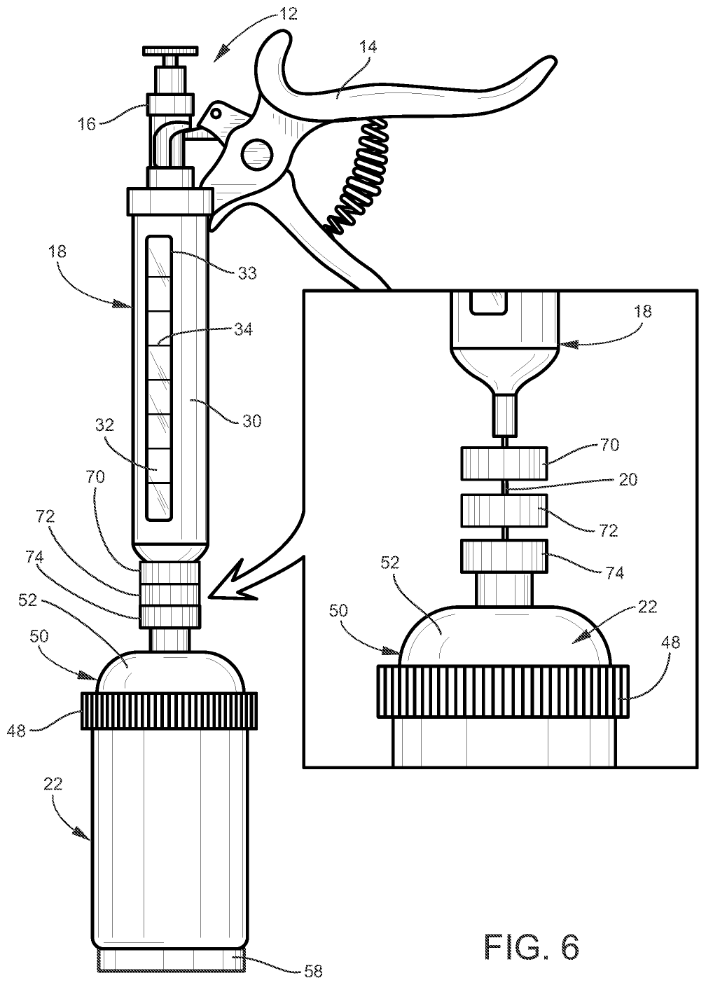

FIG. 6 illustrates an alternative apparatus for releasably connecting medicine disbursement assembly 18 to medicine container assembly 22. In this embodiment, a first donut like magnetic element 70 is sttached to the medicine disbursement assembly 18. A second donut like magnetic element 72 is positioned adjacent to element 70, and a third donut like magnetic element 74 is attached to form the end cap portion of the medicine container 22. Needle 22 may be inserted into the end cap portion 74 as described previously with respect to FIGS. 3 and 4. The magnetic attraction between elements 70, 72, and 74 provides a releasable support connection between disbursement assembly 18 and container assembly 22. For shorter needles, magnet element 72 may be removed so that the magnetic connection is between elements 70 and 74.

The present invention may be embodied in other forms without departing from the spirit and the essential attributes thereof, and accordingly, reference should be made to the appended claims, rather than to the foregoing specification, as indicative of the scope of the invention.

What is claimed is:

1. A liquid medicine disbursement system providing temperature maintenance for an extended period of time before the administration of a medicine, the medicine disbursement system comprising:
a medicine disburser;
said medicine disburser including a linearly movable plunger assembly and an insulated housing, said insulated housing having opposite ends, said plunger assembly including a plunger, and said plunger being linearly movable within said insulated housing;
a needle operatively connected to one end of said insulated housing; said plunger being operatively connected to the opposite end of said insulated housing, and said plunger assembly forcing medicine through the needle for delivery into a desired target;
said insulated housing including an insulation layer which surrounds the plunger assembly; and
the plunger assembly and the insulation layer providing a closed insulation compartment and pathway for temperature maintenance of liquid medicine for an extended period of time before delivery of the medication through the needle.

2. The liquid medicine disbursement system according to claim 1 further including an insulated medicine container, said medicine container including an insulation layer which surrounds the medicine container, the medicine container includes an end cap portion, the needle being selectively insertable into and retractable from the end cap portion, and the medicine container insulation layer providing temperature maintenance of liquid medicine for an extended period of time.

3. The liquid medicine disbursement system according to claim 2 further including the medicine disburser insulation layer and the medicine container insulation layer being located between opposed insulation walls.

4. The liquid medicine disbursement system according to claim 3 further including the insulation layers having sealed vacuum layers between the opposed insulation walls.

5. The liquid medicine disbursement system according to claim 2 further including a first attachment means secured to the medicine container for permitting the medicine container to be selectively attached at a desired location.

6. The liquid medicine disbursement system according to claim 5 further including a second attachment means connected to the ends of the medicine disburser and to the medicine container for permitting a releasable end to end connection between the medicine disburser and the medicine container.

7. The liquid medicine disbursement system according to claim 1 further including an elongated visibility section located along the length of the insulated housing and spaced apart indicators positioned along the length of the visibility section.

8. The liquid medicine disbursement system according to claim 7 further including a sensor operably connected to the plunger to sense the location of the plunger within the insulated housing and the temperature of the medicine.

9. A liquid medicine disbursement system providing temperature maintenance for an extended period of time before the administration of a medicine, the medicine disbursement system including:
a medicine disburser;
said medicine disburser including a linearly movable plunger assembly and an insulated housing, said insulated housing having opposite ends, said plunger assembly including a plunger, and said plunger being linearly movable within said insulated housing;
a needle operatively connected to one end of said insulated housing; said plunger being operatively connected to the opposite end of said insulated housing, and said plunger assembly forcing medicine through the needle for delivery into a desired target;
said insulated housing including a medicine disburser insulation layer which surrounds the plunger assembly;
a medicine container, said medicine container including an insulation layer and said medicine container insulation layer surrounding the medicine container, the medicine container includes an end cap portion, and the needle being selectively insertable into and retractable from the end cap portion;
the medicine disburser insulation layer and the medicine container insulation layer providing temperature maintenance of liquid medicine for an extended period of time; and
attachment means for releasably attaching the medicine disburser to the medicine container and for permitting an end to end positioning of the medicine disburser relative to the medicine container.

10. The liquid medicine disbursement system according to claim 9 further including the medicine disburser insulation layer and the medicine container insulation layer being located between opposed insulation walls.

11. The liquid medicine disbursement system according to claim 10 further
including the insulation layers having sealed vacuum layers between the opposed insulation walls.

12. The liquid medicine disbursement system according to claim 9 further including a first attachment means secured to the medicine container for permitting the medicine container to be selectively attached at a desired location.

13. The liquid medicine disbursement system according to claim 12 further including a second attachment means connected to the ends of the medicine disburser and to the medicine container for permitting a releasable end to end connection between the medicine disburser and the medicine container.
